(19) United States
(12) Patent Application Publication
(10) Pub. No.: US 2019/0107538 A1
(43) Pub. Date: Apr. 11, 2019

(54) EVALUATION OF THE SEVERITY OF PATIENTS WITH FLAVIVIRUS INFECTION BY BLOOD HYALURONAN LEVELS AND THERAPEUTIC AGENTS TO BLOCK THE HYALURONAN

(71) Applicant: Kaohsiung Medical University, Kaohsiung City (TW)

(72) Inventors: Yen-Hsu Chen, Kaohsiung City (TW); Chun-Yu Lin, Kaohsiung City (TW); Paraskevi Heldin, Uppsala (SE)

(73) Assignee: Kaohsiung Medical University, Kaohsiung City (TW)

(21) Appl. No.: 15/881,350

(22) Filed: Jan. 26, 2018

(30) Foreign Application Priority Data
Oct. 5, 2017 (TW) 106134411

(51) Int. Cl. G01N 33/569; A61P 31/14; A61K 31/352; C12N 15/113; A61K 39/395; A61K 38/47

(52) U.S. Cl. CPC G01N 33/56983; A61P 31/14; A61K 31/352; C12N 15/1138; C12N 2310/14; A61K 38/47; G01N 2400/40; G01N 2469/00; G01N 2800/26; A61K 39/3955

(57) ABSTRACT

The present invention discloses a method to determine whether a warning sign will occur in a subject with the Flavivirus infectious illness, and a pharmaceutical composition. The method includes steps of measuring the level of serum hyaluronan of the subject, and determining that the warning sign will occur in his/her illness course when the level is higher than or equal to 70 ng/mL. It is identified in the invention that the level of the serum hyaluronan is an excellent predicator for the severity of the Flavivirus infectious illness. The pharmaceutical composition is used for blocking the serum hyaluronan of the Flavivirus-infected subjects to prevent inflammation, and includes a therapeutically effective amount of a 4-methyl umbelliferone sodium salt, a CD44 siRNA, an anti-CD44 antibody or a hyaluronidase, wherein the therapeutically effective amount is an effective blood concentration of the 4-methyl umbelliferone sodium salt, the CD44 siRNA, the anti-CD44 antibody, and the hyaluronidase of the subject being in a range from 0.05 mM to 5 mM, 1 nM to 100 nM, 5 μg/ml to 500 μg/ml and 0.5 unit/mL to 50 units/mL, respectively.

### CROSS-REFERENCE TO RELATED APPLICATION AND CLAIM OF PRIORITY

This application claims the benefit of Taiwan Patent Application No. 106134411, filed on Oct. 5, 2017, at the Taiwan Intellectual Property Office, the disclosures of which are incorporated herein in their entirety by reference.

### FIELD OF THE INVENTION

The present invention is related to a determination method and a pharmaceutical composition. In particular, the present invention is related to a method to determine whether a warning sign will occur in a subject with a Flavivirus infectious illness, and a pharmaceutical composition for blocking the serum hyaluronan (HA) in Flavivirus-infected patients.

### BACKGROUND OF THE INVENTION

The viruses in the Flaviviridae family are single-stranded linear RNA viruses. Humans and other mammals are their natural hosts, and the viruses are spread widely via arthropods (e.g. ticks and mosquitoes). The Flaviviridae family is classified into four genera, including Flavivirus, Pestivirus, Hepacivirus and Pegivirus, and over 100 species. The Flavivirus genus includes the well-understood yellow fever virus, Japanese encephalitis virus (JEV), dengue virus, West Nile virus and Zika virus. The Pestivirus genus includes bovine viral diarrhea virus. The Hepacivirus genus is classified into 14 species which are abbreviated as the hepatitis viruses A, B, C . . . through N, wherein the well-known one is the hepatitis C virus. The Pegivirus genus is classified into 11 species which are abbreviated as the hepatotropic viruses A, B, C . . . through K.

Dengue fever, which mainly occurs in the subtropical and tropical countries, is an acute infectious disease caused by the dengue virus, and spread to humans via mosquitoes, e.g. Aedes aegypti and Aedes albopictus. The dengue viruses are classified into four types, i.e. I, II, III and IV, according to different virus serotypes, and each serotype has the abilities of infection and pathogenesis.

The latency period for the dengue fever is typically about 3 days to 8 days (the longest one being up to 14 days). The period between the day before illness and five days after illness is called “the communicability period” or “viremia”. Dengue viruses exist in the patient's blood. Mosquitoes, A. aegypti or A. albopictus, will take up the dengue viruses if the patients are bitten by the mosquitoes during the communicability period. The dengue viruses enrich inside the mosquitoes during an 8-to-12-day multiplication, and the mosquitoes bite other people causing the dengue viruses to spread.

Some patients have mild syndromes during the dengue fever infection, and some do not even appear to show any symptoms. The typical symptoms of dengue fever include the phenomena such as a sudden fever higher than 38° C., headache, posterior eye pain, muscle soreness, arthralgia, rash, drowsiness, restlessness, liver enlargement, the increased vascular permeability, plasma leakage, bleeding, severe lethal bleeding and so on. Furthermore, if patients are infected with different dengue virus serotypes (DENY) for a consecutive two times, in particular the patients who are infected with the DENY-1 firstly followed by the DENV-2 or DENV-3 infection, or the patients who are infected with the DENV-3 firstly followed by the DENV-2 infection, the possibility of causing the dengue hemorrhagic fever (DHF) and dengue shock syndrome (DSS) is significantly increased to result in more severe clinical syndromes, such as shock or severe complications. Mortality is up to more than 20% if timely treatment is absent.

There is no sovereign remedy for treating dengue fever. In the prevention of dengue fever, Sanofi Pasteur (Lyon, France) developed the first dengue fever vaccine (a tetravalent attenuated live vaccine) in the world for people between the ages of 9 to 45 years old. However, this vaccine generated poor protection against DENV-2. In addition, GlaxoSmithKline (GSK, Brentford, the United Kingdom) also developed a deactivated vaccine, TDEN-PIV, that progressed to phase II clinical trials.

The World Health Organization (WHO) classified dengue fever patients into groups A, B and C depending on the occurrence of “warning sign” or not (Dengue Guidelines for Diagnosis, Treatment, Prevention and Control, 2009, World Health Organization, ISBN 978 92 4 154787 1), rather than the simple and traditional classification for dengue fever patients and DHF patients. The warning sign means that a dengue fever patient shows abdominal pain or abdominal tenderness, persistent vomiting, clinical fluid accumulation (e.g. ascites, pleural effusion and so on), mucosal bleed, lethargy, restlessness, the liver enlargement more than 2 cm, an increase in the hematocrit (HCT) concurrent with a rapid decrease in the platelet count, while keeping in mind co-existing conditions (e.g. diabetes, renal failure, chronic hemolytic disease, obesity, pregnancy, infancy, and old age), and adverse social circumstances (such as living alone, or living far from hospital).

Among these, patients in group A do not have neither “warning signs”, co-existing conditions nor adverse social circumstances; patients in group B have “warning signs”, and have the features of co-existing conditions and/or adverse social circumstances; and patients in group C have severe plasma leakage with shock and/or fluid accumulation with respiratory distress, severe bleeding, and/or organ impairment (such as liver function impairment, central nervous system impairment, heart failure, renal failure, cardiomyopathy, encephalopathy, encephalitis and so on).

In the treatment, the patients in group A are monitored at home for disease progression; the patients in group B accept hospitalization; and the patients in group C need an emergency remedy or are transferred to the hospital with good equipment and experienced physicians and nurses.

The classification of dengue cases by the WTO (2009 Edition) was established to provide appropriate triage to assist the clinical remedy, so as to study the mechanism of the disease and evaluate medical intervention (such as intravenous dehydration, new anti-virus drugs and vaccines and so on) in the future.

It is therefore the Applicant's attempt to deal with the above situation encountered in the prior art.

### SUMMARY OF THE INVENTION

To assist doctors to evaluate the subsequent illness course of patients with a Flavivirus infectious illness at his/her early stage of illness, and to adopt the appropriate treatment or therapy, novel and progressive techniques are developed in the present invention.

The present invention discloses a method to determine whether a warning sign will occur in a subject with a Flavivirus infectious illness, and the method includes steps of: (a) providing a serum of the subject; (b) measuring a level of a hyaluronan in the serum; and (c) determining that the warning sign will occur in an illness course of the subject when the level is higher than or equal to 70 ng/mL.

In one embodiment, the subject is a human, and the period that the human is infected with a Flavivirus virus is at an early stage of the illness of the human. In one embodiment, the Flavivirus virus is a species including a yellow fever virus which causes the human to suffer from yellow fever, a Japanese encephalitis virus (JEV) which causes the human to suffer from Japanese encephalitis, a dengue virus which causes the human to suffer from dengue fever, a West Nile virus which causes the human to suffer from West Nile fever, and a Zika virus which causes the human to suffer from a Zika virus infection.

In one embodiment, the human with dengue fever shows the warning sign in the illness course, and the warning sign includes abdominal pain or abdominal tenderness, persistent vomiting, clinical fluid accumulation, mucosal bleed, lethargy, restlessness, liver enlargement of more than 2 centimeters (cm), and the increase in a hematocrit (HCT) concurrent with a rapid decrease in a platelet count.

In one embodiment, the step (b) is performed in a well of a microplate where an aggrecan is coated thereon already, and the step (b) further includes: (b1) adding the serum to cause the hyaluronan which may exist in the serum to be bound to the aggrecan; (b2) adding a biotinylated-proteoglycan to be bound to the hyaluronan; (b3) adding a streptavidin-conjugated enzyme to be bound to the biotinylated-proteoglycan; (b4) adding a substrate to react with the streptavidin-conjugated enzyme to generate a chemiluminescent reaction; and (b5) determining an optical density at 450 nm to calculate the level of hyaluronan.

The present invention further discloses a pharmaceutical composition for blocking a hyaluronan in a serum of a subject to prevent an inflammation thereof, wherein the subject was identified to suffer from a Flavivirus infection. The pharmaceutical composition includes: a pharmaceutically effective amount of a 4-methyl umbelliferone sodium salt.

In one embodiment, the hyaluronan has a level in the serum of the subject higher than or equal to 70 ng/mL to indicate that a warning sign will occur in an illness course of the subject. In one embodiment, the pharmaceutical composition further includes a pharmaceutically acceptable carrier, an excipient, a diluent, or an adjuvant.

In one embodiment, the subject is a human, and the therapeutically effective amount is an effective blood concentration of the 4-methyl umbelliferone sodium salt of the human being in a range from 0.05 mM to 5 mM.

The present invention further discloses a method for treating a subject suffering from a Flavivirus infection, including: administering to the subject a pharmaceutical composition including one selected from the group consisting of a first pharmaceutically effective amount of a 4-methyl umbelliferone sodium salt, a second pharmaceutically effective amount of a CD44 small interfering RNA (siRNA), a third pharmaceutically effective amount of an anti-CD44 antibody, a fourth pharmaceutically effective amount of a hyaluronidase and a combination thereof.

In one embodiment, the Flavivirus infection causes the subject to have an illness being one selected from the group consisting of yellow fever, Japanese encephalitis, dengue fever, West Nile fever, a Zika virus infection and a combination thereof. In one embodiment, the subject is a human. In one embodiment, the first pharmaceutically effective amount is a first effective blood concentration of the 4-methyl umbelliferone sodium salt of the human being in a first range from 0.05 mM to 5 mM. In one embodiment, the second pharmaceutically effective amount is a second effective blood concentration of the CD44 siRNA of the human being in a second range from 1 nM to 100 nM. In one embodiment, the third pharmaceutically effective amount is a third effective blood concentration of the anti-CD44 antibody of the human being in a third range from 5 μg/mL to 500 μg/mL. In one embodiment, the fourth pharmaceutically effective amount is a fourth effective blood concentration of the hyaluronidase of the human being in a fourth range from 0.5 unit/mL to 50 units/mL.

The present invention further discloses a usage of a 4-methyl umbelliferone sodium salt, a CD44 siRNA, an anti-CD44 antibody or a hyaluronidase to be prepared as a pharmaceutical composition for treating a subject suffering from a Flavivirus infection to have an illness, and the Flavivirus and the illness correlated to the Flavivirus are described above.

### BRIEF DESCRIPTION OF THE DRAWINGS

The objectives and advantages of the present invention will become more readily apparent to those ordinarily skilled in the art after reviewing the following detailed descriptions and accompanying drawings.

FIG. 1 illustrates a diagram showing a receiver operating characteristic (ROC) curve for the level of serum hyaluronan (SA) of the dengue fever patients at the early stage of the illness in the present invention.

FIG. 2 illustrates a diagram showing the ratio of the warning signs (WS)-positive to the WS-negative in the dengue fever patients during their whole illness courses in the present invention.

FIG. 3 illustrates a diagram showing the platelet (PLT) nadir (smaller than or not smaller than 50,000/l) of the dengue fever patients during their whole illness courses in the present invention.

FIG. 4 illustrates a diagram showing the blood concentration phenomenon for the increased hematocrit (HCT) level (>20%) appearing in the dengue fever patients during their whole illness courses in the present invention.

FIG. 5 illustrates a diagram showing the Akt phosphorylation in the human vascular endothelial cells being subjected to the dengue virus (DENY) nonstructural protein 1 (NS1) or the pharmaceutical composition of the present invention.

FIG. 6 illustrates a diagram showing the transwell permeability assay of human microvascular endothelial cells.

### DETAILED DESCRIPTION OF THE PREFERRED EMBODIMENTS

The present invention will now be described more specifically with reference to the following embodiments. It is to be noted that the following descriptions of the preferred embodiments of this invention are presented herein for purpose of illustration and description only; they are not intended to be exhaustive or to be limited to the precise form disclosed.

### Definition

The term “Flavivirus” herein refers to the viruses which include, but are not limited to, yellow fever virus, Japanese encephalitis virus (JEV), dengue virus, West Nile virus and Zika virus.

The illnesses of the dengue fever patients herein are classified into “dengue fever”, “severe dengue”, “dengue hemorrhagic fever (DHF)”, “dengue shock syndrome (DSS)” and so on according to the clinical manifestations or examination results.

The definition of the term “severe dengue” herein conforms to one or more items as follows: severe plasma leakage with shock, severe plasma leakage with fluid accumulation and respiratory distress, severe bleeding evaluation, severe organ impairment, glutamic oxaloacetic transaminase (GOT) or glutamic pyruvic transaminase (GPT) ≥1,000 IU/L in the liver, consciousness impairment in the central nervous system, heart failure or others.

According to the classification of the dengue cases by the WHO (2009 Edition), the term “warning sign” herein is defined as that dengue fever patients show abdominal pain or abdominal tenderness, persistent vomiting, clinical fluid accumulation, mucosal bleeding, lethargy, restlessness, liver enlargement of more than 2 cm, and an increase in the hematocrit (HCT) concurrent with a rapid decrease in the platelet count.

The term “yellow fever” herein means an illness that a subject is infected with the yellow fever virus, and the symptoms of the yellow fever include fever, abnormal liver functions, outbreak of chills, headache, backache, muscle pain throughout the body, anorexia, poor appetite, vomiting, and so on. A number of yellow fever patients enter into a poisoning stage after a relief for several hours to one day, and the symptoms such as fever, icterus, albuminuria and bleeding occurs. Even, liver or renal failure occurs to cause anuria (anuresis).

The term “Japanese encephalitis” herein means an illness that a subject is infected with the Japanese encephalitis virus (JEV), and the symptoms of the Japanese encephalitis includes fever, diarrhea, headache or vomiting. The clinical manifestation of the patients with mild symptoms is aseptic meningitis or the fever of unknown origin, and the severe patients show the worsening consciousness conditions, general weakness, high fever, partial neurological disability, dyskinesia, Parkinson's syndrome, obnubilation, coma or death.

The term “West Nile fever” herein means an illness that a subject is infected with the West Nile virus, and the symptoms include fever, headache, fatigue, arthralgia, muscle pain, rash, lymphatic enlargement, gastrointestinal disorder. The severe patients have symptoms such as cephalomeningitis, encephalitis, acute asthenic paralysis syndrome and so on.

The term “Zika virus infection” herein means an acute infection that a subject is infected with the Zika virus, and its latency period is about 3 days to 7 days (the longest one being up to 12 days). The classic symptoms are fever with rash, arthralgia or conjunctivitis (pinkeye), headache, muscle pain, posterior eye pain and so on, and even the nervous system complications (such as Guillain-Barré syndrome (GBS)) or immune system complications (such as idiopathic thrombocytopenic purpura (ITP)) may occur. If a pregnant woman is infected with Zika virus, she may deliver a newborn with neurological abnormality (such as microcephaly).

In the embodiment of the present invention, the patients who are infected with dengue virus and identified to have dengue fever are the population, and the levels of hyaluronan in the sera of the patients are measured and statistically calculated to define that the warning sign will occur in the illness course of the patients when the level is higher than or equal to 70 ng/mL. Because the diseases caused by the yellow fever virus, JEV, West Nile virus and Zika virus (where all belong to the Flavivirus) also make patients have symptoms similar to dengue fever, the technique in the present invention can also be used in patients who suffer from yellow fever, Japanese encephalitis, West Nile fever and Zika virus infection.

### Embodiment 1

Experimental Methods:

To determine whether a warning sign will occur in a patient with a Flavivirus infectious illness, the level of SA in the patient was determined using the Hyaluronan DuoSet® ELISA development system (Cat. No. DY3614-05, R&D systems, Inc., U.S.A.). The skilled person in the art can implement Embodiment 1 in view of the manufacturer's instructions of the Hyaluronan DuoSet® ELISA development system, or perform the experiments by preparing the materials and reagents by himself/herself and using the same experimental methods.

First, a 96-well microplate for the enzyme-linked immunosorbent assay (ELISA) was prepared as follows. The hyaluronan capture reagent (i.e. recombinant human aggrecan) was diluted to a working concentration in the phosphate-buffered saline (PBS, 137 mM NaCl, 2.7 mM KCl, 8.1 mM Na₂HPO₄, 1.5 mM KH₂PO₄, pH 7.2-7.4, 0.2 μm filtered) without carrier protein. The 96-well microplate with 100 μL per well of the diluted hyaluronan capture reagent was immediately coated. The microplate was sealed and incubated overnight at room temperature.

Each well was aspirated and washed with a wash buffer (0.05% Tween® 20 in PBS, pH 7.2-7.4), the process was repeated two times for a total of three washes. In detail, each well was washed by filling with the wash buffer (400 μL) using a squirt bottle, a manifold dispenser or an autowasher. Complete removal of liquid at each step was essential for good performance. After the last wash, any remaining wash buffer was removed by aspirating or by inverting the microplate and blotting it against clean paper towels. The microplate was blocked by adding 300 μL of a reagent diluent (5% Tween® 20 in PBS, pH 7.2-7.4, 0.2 μm filtered) to each well. The microplate was incubated at room temperature for a minimum of 1 hour. The aspiration/wash was repeated. The microplate was now ready for the sample addition and the ELISA experiment.

A 100-μL sample or hyaluronan standards in the reagent diluent (or an appropriate diluent) per well was added to the well. An adhesive strip was covered on the microplate, and the microplate was incubated for 2 hours at room temperature. The aspiration/wash was repeated. A 100-μL biotinylated detection reagent (i.e. the biotinylated recombinant human proteoglycan) which was diluted in the reagent diluent was added to each well. A new adhesive strip was covered on the microplate, which was further incubated for 2 hours at room temperature. A 100-μL working dilution of streptavidin-horseradish peroxidase (HRP) was added to each well. The microplate was covered and incubated for 20 minutes at room temperature. Placing the microplate in direct light was avoided. The aspiration/wash was repeated. A 100-μL substrate solution (1:1 (v/v) mixture of color reagent A (H₂O₂) and color reagent B (tetramethylbenzidine)) was added to each well, and the microplate was incubated for 20 minutes at room temperature. Placing the microplate in direct light was avoided. A 50-μL stop solution (2 N H₂SO₄) was added to each well. The microplate was gently tapped to ensure thorough mixing. The optical density of each well was determined immediately using a microplate reader set to 450 nm.

A six point standard curve of the hyaluronan standard using 3-fold serial dilutions in the reagent diluent was recommended. Thus, the concentration of the hyaluronan standard was 90, 30, 10, 3.33, 1.11 and 0.370 ng/mL. A standard curve was plotted based on the concentration of the hyaluronan standard and its average optical density.

Experimental Results:

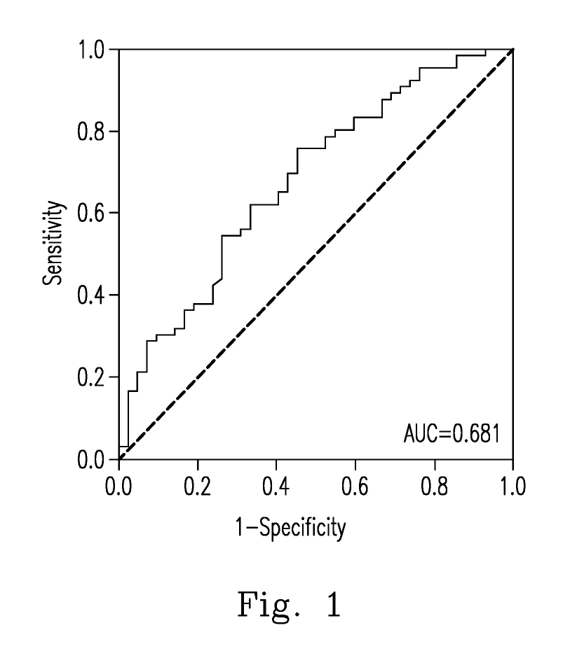

Please refer to FIG. 1, which illustrates a diagram showing a receiver operating characteristic (ROC) curve for the level of SA in the dengue fever patients at the early stage of the illness in the present invention. When the serum sample of each dengue fever patient (n=108) was measured, a critical value whether the warning sign will occur was precisely determined by the ROC curve depending on whether the patients show the warning sign. As shown in FIG. 1, an area under curve (AUC) is 0.681 (95% confidence interval (C.I.)=0.58-0.78, p value=0.002), the best critical value is 70.06 ng/mL, the sensitivity is 0.758, and the 1-specificity is 0.548. Therefore, in the subsequent analysis, the level of SA which is higher than or equal to 70 ng/mL is the critical value for determining whether the warning sign will occur in the whole illness course of the patient.

Please refer to Table 1, which is the univariate analysis of factors associated with the dengue fever patients presented with warning signs (WS) throughout the illnesses (n=108). The levels of hyaluronan in the sera of the dengue fever patients (n=108) were measured during the febrile phase (at early illness stage), and their illness progression was traced until recovery. If the level of SA in the dengue fever patient is higher than or equal to 70 ng/mL during the febrile phase, the possibility that they show “the warning sign” in whole illness course is 3.78 times higher than the patients with the level lower than 70 ng/mL (p=0.003, 95% C.I.=1.65-8.66). The results of the univariate analysis in Table 1 also showed that the age ≥65 years, the secondary dengue viral infection, and the level of SA ≥70 ng/mL (at the febrile phase) are prediction factors for predicting whether the dengue fever patients belong to the “WS-positive” group. Furthermore, important prediction factors (i.e. patient's age, and secondary infection) were calibrated using multivariate analysis, and the results still showed that the level of SA ≥70 ng/mL in the dengue fever patient at the early stage (the febrile phase) is an independent prediction factor for predicting whether the warning sign will occur in the illness course of the patient (referring to Table 2). Therefore, depending on the level of SA (≥ or <70 ng/mL) in the dengue fever patient at the early illness stage, doctors can determine whether the warning sign will occur in his/her illness course, and further evaluate the patient's subsequent conditions so as to provide him/her with the appropriate remedy or treatment.

TABLE 1
Univariate analysis of factors associated with
dengue fever patients presented with warning
signs (WS) throughout the illnesses (n = 108)

| | No. (%) of patients | | | |
|---|---|---|---|---|
| | WS- | WS- | Odds ratio | |
| | positive | negative | (95% Confidence | p- |
| Variables | (n = 66) | (n = 42) | interval) | value |

Demographic characteristics

| | | | | |
|---|---|---|---|---|
| Gender, female | 35 (53.0) | 19 (45.2) | 1.37 (0.63-2.97) | 0.554 |
| Age ≥ 65 years | 34 (51.5) | 10 (23.8) | 3.40 (1.44-8.02) | 0.008 |
| Secondary | 43 (65.2) | 17 (40.5) | 2.75 (1.24-6.10) | 0.020 |
| infection | | | | |

Comorbidities

| | | | | |
|---|---|---|---|---|
| Diabetes | 16 (24.2) | 7 (16.7) | 1.60 (0.60-4.30) | 0.486 |

Laboratory data

| | | | | |
|---|---|---|---|---|
| Thrombo- | 26 (39.4) | 11 (26.2) | 1.83 (0.79-4.27) | 0.230 |
| cytopenia | | | | |
| (first sampling) | | | | |
| SA (febrile | 50 (75.8) | 19 (45.2) | 3.78 (1.65-8.66) | 0.003 |
| phase) ≥ 70 ng/ml | | | | |

TABLE 2
Multivariate analysis of the serum samples
of the dengue fever patients (n = 108)

| | Adjusted odds ratio | p- |
|---|---|---|
| Variable | (95% CI) | value |
| Age ≥ 65 years | 2.01 (0.78-5.21) | 0.151 |
| Secondary infection | 2.00 (0.84-4.76) | 0.118 |
| Serum HA (febrile phase) ≥70 ng/ml | 2.80 (1.15-6.80) | 0.023 |

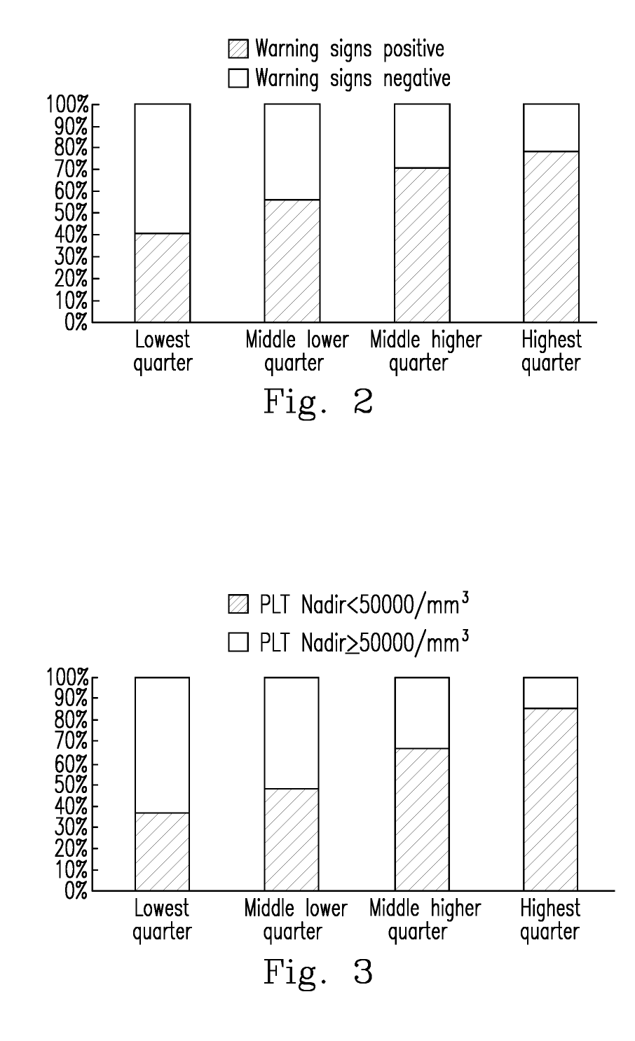

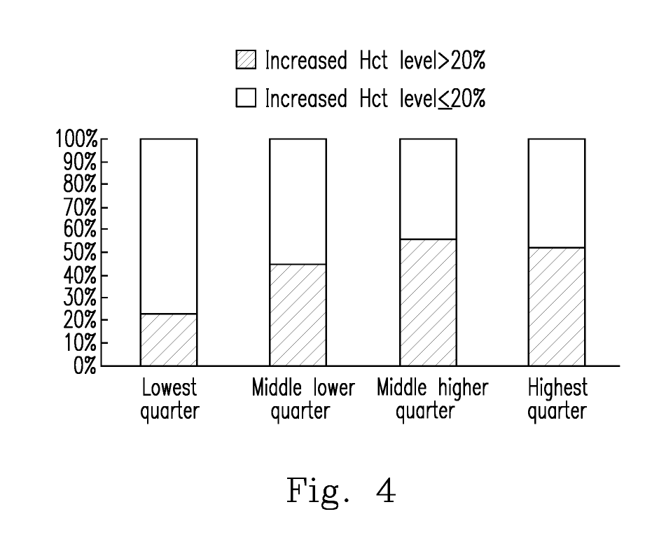

Please refer to FIG. 2 to FIG. 4, which illustrates diagrams showing (a) the ratio of the warning signs (WS)-positive to the WS-negative, (b) the platelet (PLT) nadir (< or ≥50,000/l), and (c) the blood concentration phenomenon for the increased hematocrit (HCT) level (>20%) in the dengue fever patients during their whole illness course in the present invention. These 108 dengue fever patients were classified into four groups (i.e. lowest quarter, middle lower quarter, middle higher quarter, and highest quarter) depending on the levels of SA at their early illness stage (the febrile stage). It can be known from FIGS. 2 to 4 that, compared to the group with the lower level of SA, the group with the higher level of SA has a high possibility of having the warning sign in the whole illness course, and the PLT nadir <50,000/μl and the increased hematocrit (HCT) level (>20%).

### Embodiment 2

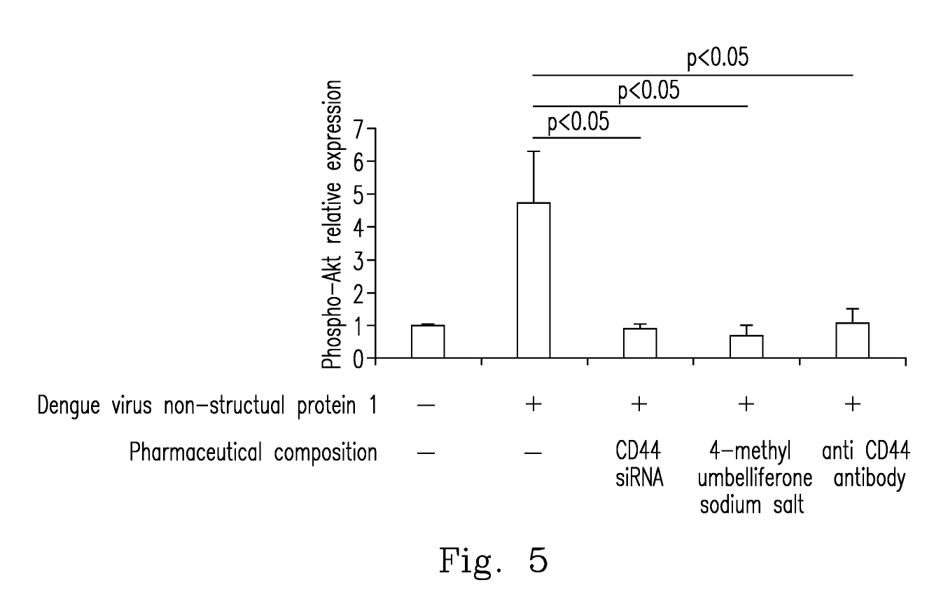

Based on whether the warning sign will occur in the illness course of the patent with the level of SA >70 ng/mL, a pharmaceutical composition for blocking the SA in the patient to prevent inflammation is disclosed in this Embodiment. The pharmaceutical composition includes a pharmaceutically effective amount of 4-methyl umbelliferone sodium salt, wherein the therapeutically effective amount is an effective blood concentration of 4-methyl umbelliferone sodium salt of the patient being in a range from 0.05 mM to 5 mM. The pharmaceutical composition may further include a pharmaceutically acceptable carrier, an excipient, a diluent, or an adjuvant. Please refer to FIG. 5, which illustrates a diagram showing the Akt phosphorylation in the human vascular endothelial cells being subjected to the dengue virus (DENY) nonstructural protein 1 (NS1) or the pharmaceutical composition of the present invention. In FIG. 5, the human vascular endothelial cells (4×10⁵ cells cultivated in a 60-mm dish) were treated with a nonstructural protein 1 (3 μg/ml, Cat. No. ENZ-PRT105-0100, Enzo Life Sciences, Inc., NY, U.S.A.), and the Akt phosphorylation was significantly induced in the cells, indicating that the dengue virus results in inflammation in the human vascular endothelial cells. In addition, the human vascular endothelial cells were treated with 4-methyl umbelliferone sodium salt for 12 hours followed by the DENY NS1 treatment. Subsequently, the cellular proteins were extracted and subjected to the sodium dodecyl sulfate polyacrylamide gel electrophoresis (SDS-PAGE) to separate the proteins according to their molecular weights. Next, the proteins in the gel were transferred to a polyvinylidene difluoride (PVDF) membrane, and the phospho-Akt (p-Akt) signal on the PVDF membrane was detected using the Western blotting and the p-Akt antibody. The results showed that a range from 0.05 mM to 5 mM is the effective blood concentration of 4-methyl umbelliferone sodium salt in the dengue fever patient, indicating that 4-methyl umbelliferone sodium salt can be used to inhibit the inflammation of the human vascular endothelial cells caused by the dengue virus (referring to FIG. 5).

### Embodiment 3

The CD44 antigen, plays important roles in many biological functions (such as inflammation), is a surface glycoprotein on mammalian cells, and hyaluronan is a main molecule to bound with the CD44 antigen. Thus, an abundance of hyaluronan would bind to CD44 to influence CD44's biological functions when the level of SA in the dengue fever patient is too high. In this Embodiment, CD44 small interfering RNA (siRNA) was used to inhibit the CD44 expression of the vascular endothelial cells of the dengue fever patients, and block the combination between hyaluronan and CD44. In this Embodiment, the experimental method for inhibiting cellular protein expression using siRNA was known to the skilled person in the art, and the CD44 siRNAs were the artificially synthesized SEQ ID NO:1 (5′-GAAUAUAACC UGCCGCUUU-3′), SEQ ID NO: 2 (5′-CAAGUGGACU CAACGGAGA-3′), SEQ ID NO: 3 (5′-CGAAGAAGGU GUGGGCAGA-3′) and SEQ ID NO: 4 (5′-GAUCAACAGU GGCAAUGGA-3′). Furthermore, two, three or four siRNAs in the SEQ ID NOs: 1 to 4 may be combined to inhibit the CD44 expression of the vascular endothelial cells (4×10⁵ cells cultivated in a 60-mm dish) of the dengue fever patient. First, in view of the manufacturer's instructions of siLentFect™ Lipid Reagent (Bio-Rad laboratories AB, Sweden), the CD44 siRNA was mixed with siLentFect™ Lipid Reagent, the mixture was transfected into the human vascular endothelial cells for 24 hours followed by the DENY NS1 treatment (3 μg/ml, Cat. No. ENZ-PRT105-0100, Enzo Life Sciences, Inc., NY, U.S.A.). Subsequently, the cellular proteins were extracted and subjected to the SDS-PAGE to separate the proteins according to their molecular weights. Next, the proteins in the gel were transferred to a PVDF membrane, and the p-Akt signal on the PVDF membrane was detected using the Western blotting and the p-Akt antibody. The results showed that a range from 1 nM to 100 nM is an effective blood concentration of CD44 siRNA (SEQ ID NOs: 1 to 4) in the dengue fever patient, indicating that the CD44 siRNA can be used to inhibit the inflammation of the vascular endothelial cells in humans caused by the dengue virus (referring to FIG. 5). Therefore, the CD44 siRNA (SEQ ID NOs: 1 to 4) of the present invention can be used to prepare a pharmaceutical composition for treating inflammation in dengue fever patients.

### Embodiment 4

In this Embodiment, an anti-CD44 antibody (MA4400, Invitrogen, CA, U.S.A.) of 5 μg/ml to 500 μg/ml was used to inhibit the CD44 surface antigen of the vascular endothelial cells of the dengue fever patients. First, the human vascular endothelial cells (4×10⁵ cells cultivated in a 60-mm dish) were treated with the anti-CD44 antibody for 12 hours followed by the treatment of DENY NS1 (3 μg/ml, Cat. No. ENZ-PRT105-0100, Enzo Life Sciences, Inc., NY, U.S.A.). Subsequently, the cellular proteins were extracted and subjected to the SDS-PAGE to separate the proteins according to their molecular weights. Next, the proteins in the gel were transferred to a PVDF membrane, and the p-Akt signal on the PVDF membrane was detected using Western blotting and the p-Akt antibody. The results showed that a range from 5 μg/ml to 500 μg/ml is an effective blood concentration of anti-CD44 antibody in the dengue fever patient to inhibit p-Akt in the vascular endothelial cells of the dengue fever patients, indicating that the anti-CD44 antibody can be used to inhibit the inflammation of the vascular endothelial cells in humans caused by the dengue virus (referring to FIG. 5). Therefore, the anti-CD44 antibody of the present invention can be used to prepare a pharmaceutical composition for treating inflammation in dengue fever patients.

### Embodiment 5

Vascular leakage is an important feature in several diseases, such as septic shock, viral hemorrhagic fever, cancer metastasis and ischemia-reperfusion injuries. Thus, an in vitro endothelial permeability assay will provide insight into the establishment or progression of such diseases. The transwell permeability assays directly detect the penetration of a specific molecule, which can be detected by a spectrometer-based absorbance reader.

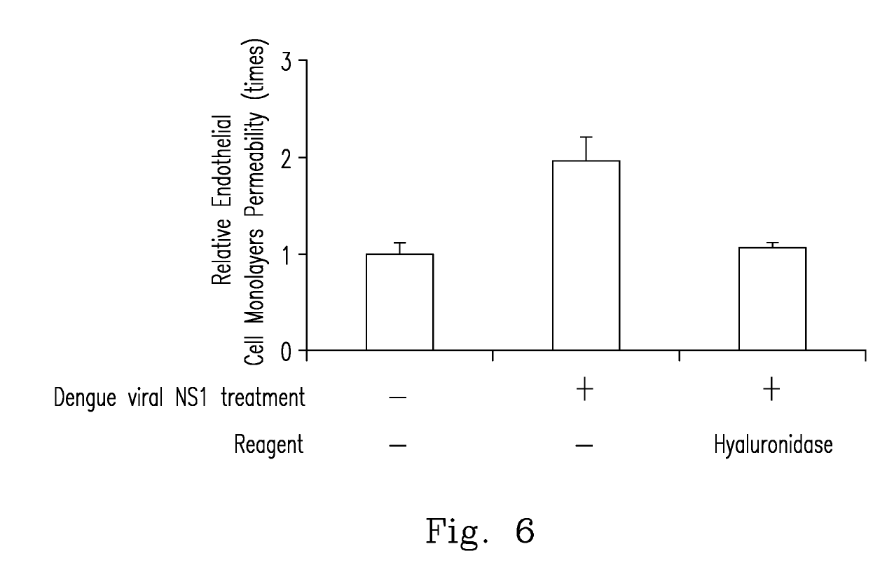

The permeability of human microvascular endothelial cells treated or not with DENV NS1 (3 μg/ml, Cat. No. ENZ-PRT105-0100, Enzo Life Sciences, Inc., NY, U.S.A.), was measured by an in vitro transwell permeability assay that mimics human endothelium in vivo (referring to http://www.bio-protocol.org/e2273). In brief, human microvascular endothelial cells (2×10⁵) were grown in a 24-Transwell® (pore size: 0.4 μm, Cat. No. 3414; Corning, Kennebunk, Me., U.S.A.) for 5 days until a confluent monolayer was formed. Next, some of the samples were treated for 6 hours with DENV NS1 protein in the absence or presence of exogenously added hyaluronidase (0.5 unit/mL to 50 units/mL), media was aspirated, and 200 μL fresh serum-free medium containing 3 μL of streptavidin-horseradish peroxidase (HRP) (GE Healthcare, Cat. No. RPN1231, UK) was added. To measure the HRP that had leaked through the endothelial layer, the inserts were moved to a new 24-well plate with 500 μl of serum-free medium, covered and placed in a 37° C. incubator for 5 minutes. Twenty microliters of medium, from each lower chamber, was transferred to a 96-well plate and analyzed for HRP activity by adding 100 μL 3,3′,5,5′-tetramethylbenzidine (TMB) substrate (Sigma-Aldrich, Cat. No. T4444, St. Louis, Mo., U.S.A.). Color development was detected by an EnSpire Multimode Reader (PerkinElmer, Upplands Vasby, Sweden) at 450 nm. The results showed that the concomitantly added hyaluronidase could reverse the DENY NS1-induced endothelial hyper-permeability significantly (referring to FIG. 6).

While the invention has been described in terms of what is presently considered to be the most practical and preferred Embodiments, it is to be understood that the invention need not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

What is claimed is:

1. A method to determine whether a warning sign will occur in a subject with a Flavivirus infectious illness, the method comprising steps of:
(a) providing a serum of the subject;
(b) measuring a level of a hyaluronan in the serum; and
(c) determining that the warning sign will occur in an illness course of the subject when the level is higher than or equal to 70 ng/mL.

2. The method according to claim 1, wherein the subject is a human, and a period that the human is infected with a Flavivirus virus is at an early stage of the illness of the human.

3. The method according to claim 2, wherein the Flavivirus virus is a species being one selected from the group consisting of a yellow fever virus, a Japanese encephalitis virus (JEV), a dengue virus, a West Nile virus and a Zika virus.

4. The method according to claim 3, wherein the yellow fever virus causes the human to suffer from yellow fever.

5. The method according to claim 3, wherein the JEV causes the human to suffer from Japanese encephalitis.

6. The method according to claim 3, wherein the dengue virus causes the human to suffer from dengue fever.

7. The method according to claim 6, wherein the human with the dengue fever shows the warning sign in the illness course, and the warning sign is one selected from the group consisting of an abdominal pain, an abdominal tenderness, a persistent vomiting, a clinical fluid accumulation, a mucosal bleed, a lethargy, a restlessness, a liver enlargement of more than 2 centimeters, an increase in a hematocrit (HCT) concurrent with a rapid decrease in a platelet count, and a combination thereof.

8. The method according to claim 3, wherein the West Nile virus causes the human to suffer from West Nile fever.

9. The method according to claim 3, wherein the Zika virus causes the human to suffer from a Zika virus infection.

10. The method according to claim 1, wherein the step (b) is performed in a well of a microplate where an aggrecan is coated thereon already, and the step (b) further comprises:
(b1) adding the serum to cause the hyaluronan which may exist in the serum to be bound to the aggrecan;
(b2) adding a biotinylated-proteoglycan to be bound to the hyaluronan;
(b3) adding a streptavidin-conjugated enzyme to be bound to the biotinylated-proteoglycan;
(b4) adding a substrate to react with the streptavidin-conjugated enzyme to generate a chemiluminescent reaction; and
(b5) determining an optical density at 450 nm to calculate the level of hyaluronan.

11. A pharmaceutical composition for blocking a hyaluronan in a serum of a subject to prevent an inflammation thereof, wherein the subject was identified to suffer from a Flavivirus infection, the pharmaceutical composition comprising:
a pharmaceutically effective amount of a 4-methyl umbelliferone sodium salt.

12. The pharmaceutical composition according to claim 11, wherein the hyaluronan has a level in the serum of the subject higher than or equal to 70 ng/mL to indicate that a warning sign will occur in an illness course of the subject.

13. The pharmaceutical composition according to claim 11, wherein the pharmaceutical composition further comprises one selected from the group consisting of a pharmaceutically acceptable carrier, an excipient, a diluent, an adjuvant and a combination thereof.

14. The pharmaceutical composition according to claim 11, wherein the subject is a human, and the therapeutically effective amount is an effective blood concentration of the 4-methyl umbelliferone sodium salt of the human being in a range from 0.05 mM to 5 mM.

15. A method for treating a subject suffering from a Flavivirus infection, comprising:
administering to the subject a pharmaceutical composition comprising one selected from the group consisting of a first pharmaceutically effective amount of a 4-methyl umbelliferone sodium salt, a second pharmaceutically effective amount of a CD44 small interfering RNA (siRNA), a third pharmaceutically effective amount of an anti-CD44 antibody, a fourth pharmaceutically effective amount of a hyaluronidase and a combination thereof.

16. The method according to claim 15, wherein the Flavivirus infection causes the subject to have an illness being one selected from the group consisting of yellow fever, Japanese encephalitis, dengue fever, West Nile fever, a Zika virus infection and a combination thereof.

17. The method according to claim 16, wherein the subject is a human.

18. The method according to claim 17, wherein the first pharmaceutically effective amount is a first effective blood concentration of the 4-methyl umbelliferone sodium salt of the human being in a first range from 0.05 mM to 5 mM.

19. The method according to claim 17, wherein the second pharmaceutically effective amount is a second effective blood concentration of the CD44 siRNA of the human being in a second range from 1 nM to 100 nM.

20. The method according to claim 17, wherein the third pharmaceutically effective amount is a third effective blood concentration of the anti-CD44 antibody of the human being in a third range from 5 μg/mL to 500 μg/mL.

21. The method according to claim 17, wherein the fourth pharmaceutically effective amount is a fourth effective blood concentration of the hyaluronidase of the human being in a fourth range from 0.5 unit/mL to 50 units/mL.


SEQUENCE LISTING (4 sequences)

SEQ ID NO: 1 - RNA, 19 nt, Artificial Sequence
  CD44 siRNA 1
gaauauaaccugccgcuuu

SEQ ID NO: 2 - RNA, 19 nt, Artificial Sequence
  CD44 siRNA 2
caaguggacucaacggaga

SEQ ID NO: 3 - RNA, 19 nt, Artificial Sequence
  CD44 siRNA 3
cgaagaaggugugggcaga

SEQ ID NO: 4 - RNA, 19 nt, Artificial Sequence
  CD44 siRNA 4
gaucaacaguggcaaugga